(19) United States
(12) Patent Application Publication
(10) Pub. No.: US 2022/0246243 A1
(43) Pub. Date: Aug. 4, 2022

(54) Computerized Fluidic System and Methods of Use for Characterization of Molecular Networks in Complex Systems with Automated Sampling, Data Collection, Assays and Data Analytics

(71) Applicant: Alan John Rosenbloom, Pittsburgh, PA (US)

(72) Inventor: Alan John Rosenbloom, Pittsburgh, PA (US)

(21) Appl. No.: 17/591,590

(22) Filed: Feb. 2, 2022

Related U.S. Application Data
(60) Provisional application No. 63/144,692, filed on Feb. 2, 2021.

(51) Int. Cl. G16B 40/10; G16B 40/20; G16B 45/00; G01N 33/50; G01N 33/68

(52) U.S. Cl. CPC G16B 40/10; G16B 40/20; G01N 33/6848; G01N 33/5005; G16B 45/00

(57) ABSTRACT

Our biology, in health and disease, is characterized by multiple cooperating molecules in highly regulated networks. Derangements of these networks can identify imminent severe worsening of disease, known as “the tipping point”. Identifying this change early has been shown to predict worsening, but also, to reveal opportunities for specific molecular therapies to halt disease progression. Unfortunately, there are no tools currently available to characterize molecular networks in humans or to see important changes coming. The current invention is a computer system linked to computer networks and data sources, to micro- and milli-fluidic sampling and assay devices. It uses advanced data analytics to examine the available data and to learn how to recognize impending trouble at a time when there are recognizable processes to block, and before it is too late for treatment.

### CROSS-REFERENCE TO RELATED APPLICATIONS

This application claims the benefit under 35 U.S.C. § 119 of U.S. Provisional Application Ser. No. 63/144,692, filed Feb. 2, 2021 which is incorporated by reference herein in its entirety.

### STATEMENT REGARDING FEDERALLY SPONSORED RESEARCH

Not applicable.

### BACKGROUND OF THE INVENTION

Fundamental molecular mechanisms underlie all the processes of biology, including both health and disease. Rather than being random, they are organized into well regulated networks of interactions. Severe illness changes healthy networks into raging torrents of interactions out of control. The change from health to severe illness can happen rapidly, as demonstrated by the Corona Virus Disease 2019 (COVID-19) pandemic. The worldwide evolution of this healthcare crisis caused an unprecedented global scientific focus on failing molecular networks. These studies showed the promise of data analytics to begin to understand these complicated networks.

In other work, it has been shown that molecular networks dynamically adjust themselves to challenges but, at some point, if the challenge is too extreme they can fail quickly and catastrophically. This has been described as their “tipping point”. The concept of “tipping point” is defined as “the point at which a series of small changes or incidents becomes significant enough to cause a larger, more important change”. Tipping points have been observed in patients who suddenly worsen. There is now considerable interest in detecting the approach of tipping points in biological networks because the severe illness that accompanies them is easier to prevent than reverse.

Animal models have demonstrated that, early in network failure, there is an opportunity to identify key molecules in the process and, blocking them can sustain the network. However, once tipping begins, the networks quickly become disorganized and too complex to identify the instigators and too late to stop all their effects. Unfortunately, the transition from treatable to untreatable can happen very quickly. This was elegantly demonstrated in an animal study of liver ischemia to be about 2 hours (Tohme S et al., J Immunol 2019; 202:268-277). Multiple studies from the sepsis research era showed similar time frames during which potential drugs could work. No successful drug was ever approved for severe sepsis despite over 100 randomized clinical trials. Taken in the light of the new data on molecular dynamics, this makes sense. There is still no way to reliably measure human networks at risk and thus no possibility to intervene at exactly the right time.

Identifying impending failure in molecular networks requires measurements of molecules to identify the process. These are called “biomarkers”. In today's medical practice, however, biomarkers are rare and outdated. For example, the biomarkers used for managing immunosuppression and secondary infection with COVID-19 (common problems) are antiquated and ineffective: sedimentation rate (discovered in 1897), C-reactive protein (1930), procalcitonin (1993), and other legacy biomarkers are routinely used. These tests have fallen far behind the high tech, high potency drugs that are given to ill COVID-19 patients. They often receive steroids and blockers of intracellular pathways and specific inflammatory molecules. These new drugs reflect decades of progress in molecular understanding, yet the diagnostic biomarker molecule tests are from the pre-molecular era. The record for reversing severe COVID-19 is not good.

In the 2000s, there have been revolutionary advances in molecular testing. Hundreds or thousands of molecules can be measured in a drop of plasma. Robotic systems can automate sample collection. Lab-on-a-chip (LOC) technologies can measure multiple molecules simultaneously. Increasingly powerful data analytics are beginning to sort out clinical and molecular network scenarios, making predictions. Progress has even been made in analytics that allow predicting some chaotic systems from initial conditions. This suggests that, even after the tipping point, when networks become more complex and chaotic, it may not be too late for guided intervention. It is time to consider linking these capabilities into practical bedside systems that can explore tipping points and human molecular networks during routine clinical care in high risk patients.

While molecular studies must eventually come to the bedside, the interpretation of this data is complicated by multiple factors. The large inter individual differences between patients, different age groups, different sexes, different genetic backgrounds, comorbidities etc. must be taken into account. Further variability accrues from treatment variation, disease severity and different time courses of illness. The demographic and clinical data can be crucial to understand the triggers for molecular events. For this reason, the present invention gathers both clinical and molecular data.

The present invention relates generally to the fields of microfluidic sampling, Lab-On-a-Chip (LOC) molecular assays, pervasive computing and data analytics. By combining these, this invention can potentially provide a platform for molecular measurements at the bedside. More importantly, by leveraging rapidly improving data analytics it may lead to practical molecular monitoring and allow identification of key molecular targets in the window of opportunity, before tipping.

### BRIEF SUMMARY OF THE INVENTION

Economic and regulatory reality must be figured early in the design process for any discovery tool that will interface with patients. The current device design has been heavily influenced by cost analyses. The following strategies were used in development: (1) Minimally invasive patient sampling is used to qualify for low risk studies to simplify device approval in research studies, (2) automation of sampling, assays and data gathering functions markedly reduces infrastructure and staffing needs for deployment, (3) self monitoring of the device for failures and transmitted alarms allows support staff to operate remotely for troubleshooting or shut down, (4) use of inexpensive disposable sample units that can be mass produced at low cost; all other parts are reusable, (5) use of reliable, well tested, off-the-shelf technology whenever possible, (6) modular components that do not require custom manufacturing; for example, the invention should interface easily with most lab-on-a-chip (LOC) or Point-of-Care (POC) assay devices with a simple connection, (7) after sample collection, adaptation to high throughput sample handling robots and sample batching is used to reduce cost of laboratory costs, (8) stackable sample cartridges minimize use of valuable freezer space.

That these practical matters will bear significantly on technology development of discovery tools like this invention is illustrated by a simple example. The barriers to innovating better molecular clinical tests are many and high. Despite the introduction of hundreds of new drugs developed over the last 6 decades, there are only six biomarker tests routinely useful in critically ill patients. The most recently introduced is Nephrocheck®, aimed at quantifying kidney injury, a frequent result of serious illness. It was introduced in 2014 after 6 years of development costing $150M. It will not be economically feasible to develop sufficient numbers of biomarkers to optimize patient care for the molecular era using this pathway. Technology that can accelerate biomarker development and approval of diagnostics while reducing cost and time while preserving patient safety is needed.

While this invention is aimed at critically ill humans, it has uses far beyond this area. Its ability to become portable and wearable opens the possibility of use in every day life to maintain health. Its embedded systems and microcomputers will allow deployment in chronic disease management, clinical pathway monitoring and dynamic clinical management at a far more sophisticated level than currently available devices. Its portability, ability for low power consumption, computerization and sample handling will allow future versions to augment genomic, epigenetic, and gut flora studies. Aside from human health uses, it can be adapted for bioprocess monitoring e.g. biologicals, drug development, cell culture for meat substitution, environmental monitoring e.g. pollution, ocean dead zones, environmental compliance, animal monitoring (livestock, pets, wildlife health) and plant monitoring (crops, deforestation, acid rain, habitat destruction, effects of global warming, etc.).

To acquire high quality samples, this invention can use technologies from the fields of micro-sampling (microdialysis, open flow microperfusion, exhaled gas sampling, iontophoresis, various body fluid sampling methods to capture saliva, sweat, tears etc.) microfluidics, lab-on-a-chip, surface chemistry, microcomputers, wireless connectivity and networking. By placing these capabilities in a single interconnected system, an interactive environment can be created that will bring the combined expertise of clinicians, data scientists and biomedical researchers to the bedside to learn from individual patients.

### BRIEF SUMMARY OF HOW THE SYSTEM WORKS

A catheter or probe is placed into a source of fluid. This could be through a simple intravenous (IV) catheter, urinary collection system, wound (natural or after surgery), organ (after a transplant or other surgery), collected by microneedles in a wearable skin patch, in sweat etc. One or more pumps collect a small amount of fluid continuously. The fluid is divided into (relatively) large and small chambers and segregated by time of collection. Each sample is time stamped. Multiple design features prevent the samples from degrading giving high quality samples. Some samples are pre-treated to make them “test friendly” for certain technologies. The entire process is automated. The only staff involvement needed is to remove filled sample cartridges and to replace them with empty ones. This could be as infrequent as once per day.

The device compartments having relatively larger samples are frozen in batches for later analysis. These go for batch testing in quality controlled settings, allowing valid comparison between different time points. These samples are important for defining overall molecular network behavior. Leveraging high throughput technologies these samples make possible hundreds or thousands of molecular measurements that can be compared over time, defining how networks evolve.

Smaller samples are shared with attached bedside assays that measure a subset of molecules that have been shown to be key in particular disease processes. Embedded, micro- or macro- or virtual computers connected with the system collect data from multiple sources to capture the clinical context of each patient (along with the molecular data from the real time assays). This data can include hundreds of data points from diagnostic tests, treatments, clinician observations, feedback from smart devices such as Ws, mechanical ventilators, cardiac monitors, pulse oximeters, local environmental sensors, etc. Decision software decides between alternate tests, sample storage conditions and testing frequency based on prediction of tipping points (molecular crises), made from ongoing and past data collection and data analytics. The molecular changes of these crises are captured in great detail by intensified sampling of specifically chosen molecules. This will improve predictive models, and allow discovery of potentially valuable biomarkers and therapeutic targets for drug development.

The system learns from every patient. Ultimately, large databases can be built and predictive algorithms optimized by studying multiple patients during routine patient care

### BRIEF DESCRIPTION OF THE SEVERAL VIEWS OF THE DRAWINGS

FIG. 1 depicts one embodiment of the sample storage component of the present invention. On the left is a diagram of the simplest system used to collect samples from a vein. Here, a peristaltic pump supplies fluid to a microdialysis probe which is located in a vein. On the right is the top half of a storage module showing 3 curled fluid storage compartments with connecting channels below them.

FIG. 2 depicts the bottom half of the sampling module. This is joined with the top half to complete the storage compartments. The photographs at the bottom depict how the device fills with sample, sequentially, from left to right, over time. A fluorescent solution is used for visibility.

FIG. 3 depicts a prototype printed circuit board (PCB) with electronics on the right. It is connected to two small PCBs on the left that have detectors to monitor the progress of the filling of the sample compartments.

FIG. 4 depicts a schematic of one simple arrangement wherein computer controlled valves divert sample fluid from the main sample stream to alternative devices, containing lab-on-a-chip (LOC) or Point-of-Care (POC) real time assays. This is directed by data and decision making software. The data is derived from clinical data, assay results and data analytic predictions. This is called smart sample sharing.

FIG. 5 depicts a diagram of one simple arrangement for sample sharing that uses valves and design features to assure that partner assays get only fresh fluid. Old fluid that accumulates in the collecting system is flushed into (round) waste wells. This avoids contamination of later assays with fluid left over from earlier assays.

FIG. 6 depicts one method of adding reagents to collected stored samples to prevent degradation and to prepare them for freezing or specialized laboratory assays. The feature pictured is an educator. This makes use of the venturi effect to draw in reagent fluid from a side well, and mixes it with the sample being collected.

FIG. 7 depicts several arrangements of the system on a patient. These range from simplified wearable systems to more complete systems, which are only partially wearable. As components continue to get smaller, the entire system will eventually be wearable. At the bottom of the figure are some selected components. From left to right: a peristaltic pump, a double glass walled refrigeration enclosure (Dewar), a minicomputer plus a power supply good for 30 hours.

FIG. 8 depicts the development of a refrigeration unit through full sized and miniaturized prototypes. This is used to chill samples to minimize degradation over time.

FIG. 9 depicts various data sources that can contribute clinical, demographic, local sensor and smart device data that can be collected and used to test and optimize smart sample sharing, bedside assays, and predictive mathematical models. FIG. 9A depicts communications with Internet of Things (IoT) devices. FIG. 9B depicts communication with smart devices such as medical support hardware. FIG. 9C depicts communication with servers for Electronic Medical Record (EMR) data and to use and update data analytics.

FIG. 9D depicts networking with the edge computing environment for rapid access to mathematical model outputs and decision making software used for smart sampling.

Optimally, the data from each patient should be given to the patient and caregivers with permissions. Using de-identification techniques data from many patients would help researchers to discover diagnostic and therapeutic leads. FIG. 10 shows an intuitive way that the data could be displayed for viewing in retrospect (so that it does not influence patient care) for learning purposes. The system can generate an encrypted timeline derived from the collected data, molecular measurements, treatments and other events of interest.

### DETAILED DESCRIPTION OF THE INVENTION

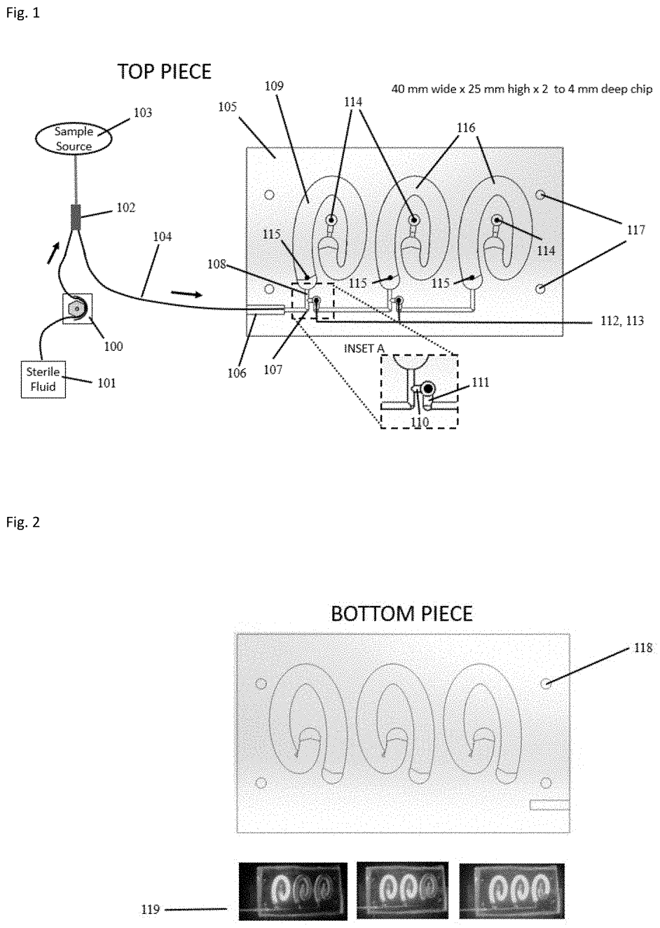

The system operation starts with sample collection using available methods of sampling to gather “liquid biopsies” of complex systems. For example, well established methods to sample blood, bile, lymph, wounds, tissues and organs and their secretions include microdialysis, iontophoresis, microneedles and open flow microperfusion. Intact fluids are easily sampled, once access is obtained. These include integumentary interstitial fluid, urine, sweat, tears, cerebrospinal fluid (CSF), intraocular fluid, prostatic secretions, lung fluids (sputum, pulmonary edema, hemorrhage), gastrointestinal fluids (saliva, secretions from the esophagus, stomach, biliary system, small and large intestine, pancreas), ascites, pericardial and pleural effusion, joint fluids, and edema fluid from multiple sites. Skin layers (epidermis, dermis, etc.) are particularly accessible because of peripheral location. Non-traditional fluids such as solvated exhaled breath condensate can also be collected. In one possible embodiment (FIG. 1), a microdialysis catheter is shown 102.

A variety of molecular and cellular components can be extracted from fluidic or solvated samples. Molecular, micro- and macroscopic components ranging from the smallest ions to the largest biomolecules, to exosomes, virus, bacteria, fungi, solid blood components such as platelets, red and white cells and cells from bone marrow and spleen, can all be sampled. In short, any soluble or non-soluble entity that can be carried away of the body with a carrier fluid or added to carrier fluid ex vivo or trapped in a hydrogel or tissue embedded permeable capsule and irrigated out can be collected by the current invention.

With portable and/or wearable systems, sampling can potentially occur over time frames from seconds to years, within or without a medical environment. Power for operation can be AC or DC current, from line supply or battery, solar, wind, water etc.

Sample collection is driven by one or more pumps. The system can use many other types of pumps with many types of force generation. A few examples include syringe pumps, peristaltic pumps, positive pressure, negative pressure, rotary, centrifugal, impeller, bubble generating, membrane/diaphragm driven, ultrasonic, electrophoretic, osmotic and many others. All can be used by this system singly or in combination.

The fluidic part of the invention is a hybrid system taking samples both for later analysis and for immediate assay. Stored samples will be later analyzed in a laboratory environment with quality control (QC). To generate real time assay results, the system will share sample fluid from the sample stream by using valves to divert the sample stream to Lab-on-a-Chip (LOC), Point-of-Care (POC) or other real time portable assay technologies. There are multiple advantages of this “assay now/assay later” combination within the invention: (1) Any researcher or device developer can easily couple their real time assay device to this system to compare their assay results (obtained in real world patients and scenarios) to the simultaneously stored sample results that benefit from processing in QC laboratories. This will accelerate validation of LOC devices. (2) a wide range of analytic technologies can be used to process the stored samples, such as protein antibody arrays, polymerase chain reaction, mass spectroscopy, chromatography etc. This range of assays far exceeds the capability of any LOC or POC device. This will allow researchers to augment their LOC assays with newly discovered molecules as needed, to improve their effectiveness. (3) The stored samples are batch handled and analyzed at the same time, by the same laboratory, with the same machine settings, standards and controls. This makes the results comparable from one sample to another. This is crucial for studies of dynamic systems over time. (3) Due to the computing power of this invention, the clinical data, plus the real time assay results can also interact with predictive and diagnostic models in real time to demonstrate the potential value of both the assays and the data analytics in a real world setting. The stored samples can help with discovery of new target molecules for future advances.

In one embodiment (FIG. 1), a standard microdialysis setup is used to collect body fluid (arrows indicate the direction of fluid flow): A peristaltic pump 100 draws sterile fluid 101, feeding it to a microdialysis catheter 102. The fluid enters the body fluid of interest 103 (such as a vein) and it equilibrates with the body fluid by diffusion and some ultrafiltration. The fluid returning from the microdialysis catheter 104. This fluid has sampled molecules from the body and is processed within the invention.

In one embodiment of the stored sample module (FIG. 1), the top piece of the module is shown 105. This is a simplified example; many other arrangements are possible. This is the top half of a sample cartridge that is designed to be removed from the system when full, to be replaced by a new, empty cartridge. The cartridge can be made from any of many polymers or types of glass. Manufacture at small or large scale can be done with many techniques, such as, but not limited to, injection molding, hot embossing, soft embossing, casting, lamination, laser ablation, traditional micro-electromechanical systems (MEMS) processes, 3D printing etc. Mold production also has many available methods.

Surface chemistry is a very important component of the current invention. Passive valves are used to distribute the fluid in the storage module; these are hydrophobic valves. These operate in this invention as follows: Fluid enters the sample collection cartridge 105 through an entry channel 106. When the fluid reaches a bend in the channel, 107, flow is directed upward into the first channel 108. Fluid flows through 108 and fills the first sample well 109. This occurs due to 3 factors: (1) the channel 108 is hydrophilic, enhancing capillary flow, (2) the channel 108 has a larger cross sectional area than channel 110 (see Inset A), favoring flow, and (3) the surfaces of the smaller channel 110 and part of the larger channel 111 (see Inset A) are superhydrophobic, impeding downstream flow, and favoring flow into the channel 108, and up into the first well 109.

Once the sample well 109 is filled, increasing pressure is created in the fluid channels 108 and 110 by continued incoming flow through entry channel 106. This back pressure forces fluid through the superhydrophobic valve 112 and rightward toward the second well channels. Once the superhydrophobic valve 112 is broken, the channel pressure falls (this is the nature of hydrophobic valves). After the second well fills, superhydrophobic valve 113 is opened by back pressure and the cycle repeats for the third and any further downstream wells.

GAS MANAGEMENT: As the sample wells fill, air escapes from the air vents 114. The geometry of the air vents 114 is narrow and their surfaces are superhydrophobic. This prevents fluid that is flowing along the superhydrophilic surfaces of the sample well from entering the air vent. Such fluid entry would foul the air vent, trapping a bubble, and creating a reduced sample size in the sample well. Pressure from bubble compression can also break the air vent hydrophobic valve, creating a leak. The superhydrophobic coating of the air escape valves 114 inhibits bubble trapping at the air outflow. Note that the air escape vents do not contact sample so they can have larger superhydrophobic areas without sample compromise.

Entry holes and ports can be on any surface. For this embodiment (FIG. 1), a side entry port for sample stream entry is shown leading into channel 106. Top surface ports are illustrated by black dots numbered 112, 114 and 115. These are for: introduction of superhydrophobic chemistry for channel superhydrophobic valves 112, 113, and for air escape vent superhydrophobic valves 114. Some ports 115 are there to allow introduction of superhydrophilic chemistry, also lyophilized reagents to be added to samples (as needed), and automated sample extraction for analysis.

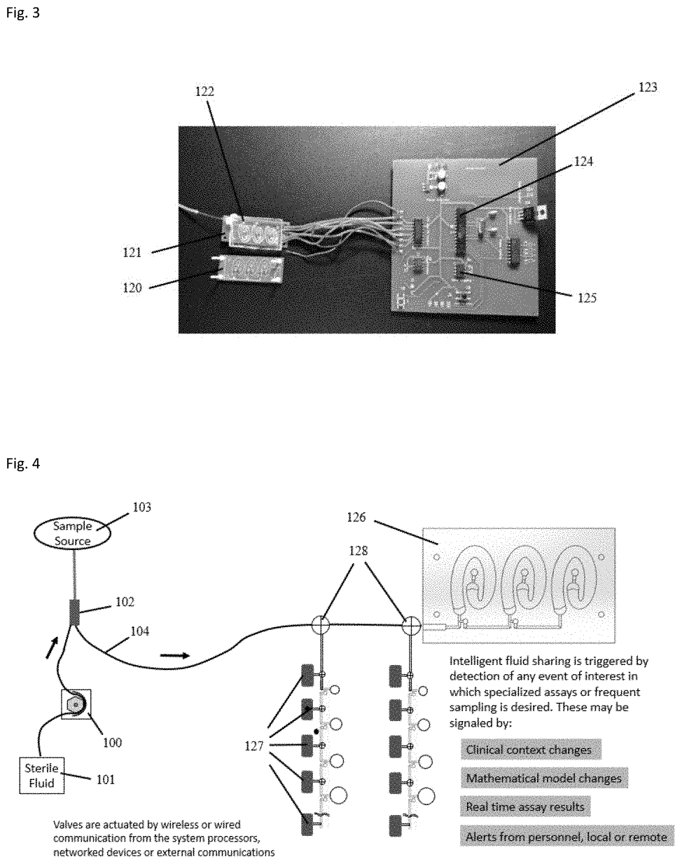

The shape of wells that store samples is important. The wells can be straight or curved, in one or more planes. However, tubular wells are preferred, as shown in 109 and the two other wells 116. Their tubular shape, combined with a superhydrophilic surface chemistry promotes a smooth, linear fill that is bubble free. The wells also empty with the same smooth removal pattern upon sample extraction and are completely emptied by automated pipetting. Furthermore, tubular wells provide spaces for detector placements that yield confirm system functioning and allow the calculation of approximate flow rates in the storage module 105 (see FIG. 3 description for more details).

Channel sizes can range from nanometer to millimeter size. In this embodiment, they are 0.5 mm diameter to make injection molding feasible. Smaller than this requires micro-computer numerical control (μCNC) for mold making, which is considerably more expensive for mass production.

Although many hydrophobic valves have been widely published, including in our early work (Yang B et al., Int. J. Nonlinear Sci Numerical Sim., 2002; 3:3-4), these rely on the hydrophobicity of the bulk material, which are usually polymers. This has disadvantages. First, it does not work well with less hydrophobic device materials. Second, hydrophobic surfaces adsorb many biomolecules, particularly proteins and lipid soluble molecules. The large surface area to volume ratio in microdevices with hydrophobic surfaces makes this a severe problem with sampling biofluids for example. Hydrophobic surface area must be carefully minimized. This problem was solved in the current invention by using superhydrophobic chemistry on surfaces (static water contact angle above 150° and contact angle hysteresis less than 5°), and exerting strict control of chemistry placement. In this way, the surface area that is hydrophobic can be reduced while the high wetting angle creates a higher energy barrier, maintaining good valve function with smaller areas of hydrophobicity.

Hydrophilic surfaces will adsorb proteins and lipid soluble molecules to some degree. Given the very small sample size and the need to measure low concentration molecules, combined with the high surface area to volume ratio in microdevices, even hydrophilic surfaces are a problem with analyte loss. This was solved in this invention by using so-called “stealth” chemistry to create superhydrophilic surfaces (static water contact angle<5° and protein adsorption<5 ng/cm²). These surfaces are used throughout the device, except for the very small areas devoted to the hydrophobic valves.

Alignment pins 117 assure accurate alignment with the mating BOTTOM PIECE of the sampling cartridge.

One embodiment of the bottom piece (FIG. 2) is shown. The bottom piece mates with the top piece to provide more well volume. The round sample well shape achieved also bends light (acts like a lens) better than a semicircular cross section. This is important for non-contact sensing (see FIG. 3 description for more details). There are matching alignment holes 118 that mate with the TOP PIECE pins. Notice that there are no matching channels on the bottom piece. This eliminates the potential for misalignment. The channel profiles, in this embodiment, are half circles, reducing inner surface area. At the bottom of FIG. 2, a sequence of photographs 119 show the sequential fill of the wells with fluorescently labeled fluid.

One method of system monitoring using non-contact detectors (FIG. 3) is shown. The basic principle is optical detection of the approach of the fluid front (time-of-flight) as the well fills linearly along a tube-like shape. This is done using 2 clamp-on printed circuit boards (PCBs); one of them 120 contains light emitting diodes (LEDs), while the other 121 (opposing) PCB contains photodiodes (PDs). The sample storage module 122 is sandwiched between them. As fluid enters the tubular well and replaces air, the index of refraction (IR) changes from 1.00 to about 1.29. The LED light is better focused by the higher IR aqueous media and the PDs detects this change. Another PCB 123 collects data from the PDs, and uses a processor chip 124 and a data logger 125 to record relevant sample data. For example, fluid arrival in the well proximal and distal sensors yields the exact beginning and end time of each sample. Also, the arrival time at various points in the system allows calculation of an approximate flow rate. This system will detect system malfunctions including, underpowered pumps (slower flow rate), pump failures, leaks and disconnects (failure of samples to arrive at expected points). This can trigger local and/or remote alarms for troubleshooting. This detection system is inexpensive and highly reliable. There are many other methods of system monitoring and obtaining fluidic flow rate in microsystems. Most of them could be used in this system.

A simplified scheme (FIG. 4) for intelligent sample sharing between the storage module 126 and real time assays 127 is shown. Many other arrangements are possible. Rows of active, computer-controlled valves 128 temporarily shunt sample to connected real time LOC assays 127. This sample sharing process can be triggered in multiple ways. For example, molecular events that are important to track continuously, such as estimators of the state of inflammation or immunosuppression, which are important systemic characteristics, could get shared samples at regular intervals based on a TIMER. Intermittent events that may indicate an interesting time to increase or change molecular measurements, such as an adverse event indicating a tipping point, could get sample sharing frequently to cover the event(s) of interest. This will have two important functions in research studies: (1) to validate predictive models that a tipping point has been predicted, and (2) to determine the key molecular initiators before the network complexity makes them impossible to recognize. Eventually, there will be a third important function: to alert caregivers that urgent and immediate action is required, such as a change in drug or other therapy before the tipping point window for action closes.

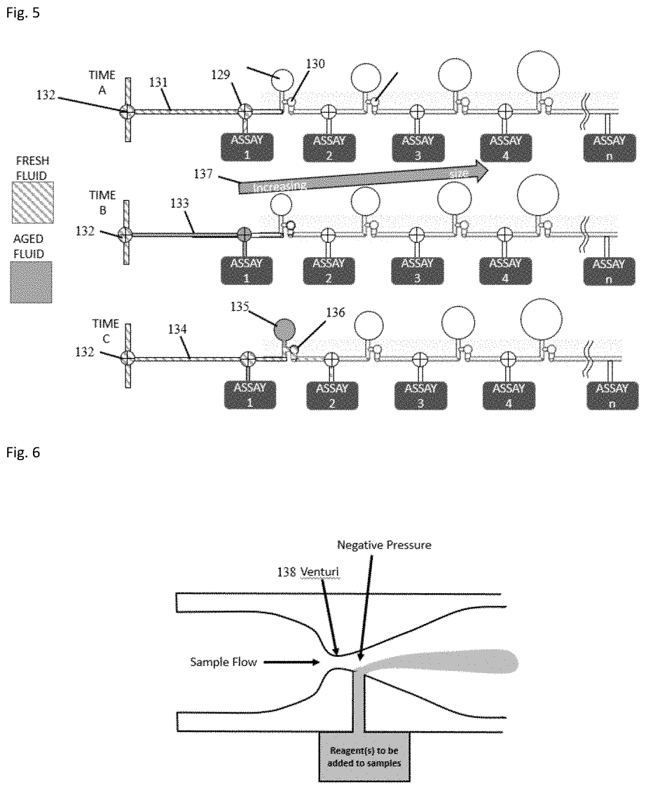

Details of one possible embodiment of sample sharing are shown (FIG. 5). Many other arrangements are possible to achieve the same results. In this embodiment, a combination of active 129 and passive 130 valves is used. This setup solves the problem of contaminating timed or intermittent samples with fluid retained in the channel system from prior sampling. To illustrate this solution, three time points are shown. At time A, fresh sample fluid 131 (hatch marked) flows into Assay 1. Once a measured amount of fluid is injected (or a sensor detects that the assay is filled), the primary control valve 132 closes and flow stops until a later time. At time B, the old fluid from the prior sampling is still in the tubing 133 (marked in grey). At time C, another sample is triggered so the primary control valve 132 opens again, and fresh sample is injected. fresh sample fluid flows in 134 (hatch marked). The fresh fluid displaces the old sample into a waste well 135.

Once the waste well 135 is filled, the pressure on the hydrophobic valve 136 increases and the hydrophobic valve 136 opens, allowing the fresh sample to flow into Assay 2. This cycle repeats until all the assays are filled. Note that the waste well sample sizes increase 137 because more of the old sample is left in the tubing due to longer path lengths for the later samples. The waste wells must be bigger for the later assays.

Some samples require reagents mixed in with them. This can be done with active pumping of a reagent into the sample stream (not shown). It can also be done with passive reagent addition. One embodiment is shown (FIG. 6). This method uses a Venturi 138 to mix the sample and the reagent fluid together. This device is commonly called an educator and it is used for multiple mixing purposes in industry and various consumer products.

The reasons for reagent addition are many. In some cases, strict attention must be paid to the sample type and the assay(s) that will analyze the sample. Many samples have fragile or labile molecules, cells or other components. These must, in some cases have chemical protectants added to them for safe storage. This may include, but not be limited to, DNase or RNase inhibitors to preserve nucleic acids, protease inhibitors to preserve proteins, anti-oxidants, cryoprotectants such as trehalose for exosomes and other analytes such as proteins, cellular cryoprotectants such as dimethylsulfoxide (DMSO), anti-coagulants to prevent clotting, optimization of anticoagulation with EDTA for lipidomic and metabolomic mass spectrography, etc.

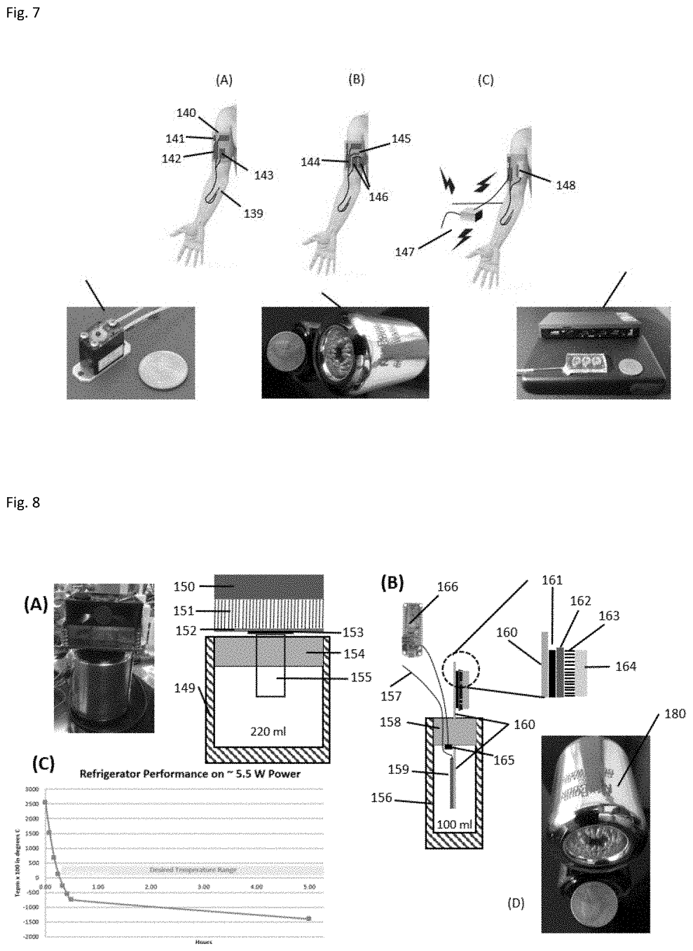

Since the components of the fluidic system are amenable to miniaturization, and computer components continue to shrink while preserving memory and processor power, the system can be made portable or wearable. One embodiment of a wearable or partially wearable portable system is shown (FIG. 7).

The basic system (FIG. 7A), is very simple, fully wearable and battery powered. In this embodiment, an intravenous microdialysis probe has been inserted into an arm vein 139, through an IV catheter. A wearable armband 140 holds the system components. A small container of sterile buffer 141 provides infusion fluid to a miniaturized peristaltic pump 142. The sterile fluid is pumped into the arm vein catheter with the microdialysis probe inside it 139. The fluid in the probe equilibrates with the patient's plasma and the fluid flows out of the probe, up into the sample storage module 143 for storage, with or without attached companion. The basic system uses a low power pump and needs minimal power and no logic. Given the constant flow rate, sample time stamps can be derived from the start time, flow rate and capacity of each sample compartment.

A more sophisticated system (FIG. 7B). A more accurate stepper motor pump 144 has replaced the DC motor pump. An example of the pump is pictured at the bottom, left. This one in this embodiment is produced by Takasago Fluidic Systems (Nagoya, Japan). A small computer module 145 has been added to drive the stepper motor with pulse width modulation (PWM). This allow variations in pumping flow rate and start/stop for pumping. The computer can collect clinical data, perform system maintenance, and provide smart sampling with attached companion assays 146.

In a high resource setting, an upgraded version (FIG. 7C) becomes portable, currently due to power constraints and bulkier equipment. A bedside module 147 has been added. The module supplies external AC wall power. There is a backup battery for interruptions in AC power. The bedside unit adds sophisticated computing and wireless capabilities with faster processors, larger storage etc. There is also a miniaturized refrigeration system for sample preservation 148. The refrigeration container (vacuum glass in this embodiment) is pictured at the bottom, middle. The computing and power modules are pictured at the bottom, right.

An embodiment of a miniaturized refrigeration module (FIG. 8) for sample preservation is pictured top right. A 220 milliliter capacity mirrored vacuum glass insulated hull 149 was used for the testing prototype (FIG. 8A). This extremely efficient container type, invented by Sir James Dewar in 1892, can have R-values up to 66 ft²*° F.*hr/BTU (square feet×degrees Farenheit×hour/British Thermal Units). Silica aerogel is an alternative that works but it is less efficient −10.3 R-value. Aerogel fractured in prototype testing. There are many less efficient options as well that can be used.

One embodiment uses a Peltier chip cooling. Heat removal from the Peltier is active in this embodiment. However, it can be augmented or replaced with passive cooling using ice or cold packs. For active heat removal there is a fan 150, atop cooling fins 151, attached to a vapor chamber 152 for rapid heat dissipation. Note that vacuum chambers spread heat with extreme efficiency, markedly increasing the cooling surface area and are ideal for refrigeration efficiency. Under the vapor chamber lies a Peltier thermoelectric chip with the hot side up 153. The chamber is sealed with polyurethane foam 154. Heat is removed from the chamber by a hollow copper pipe with capped ends 155.

A preferred embodiment is scaled down and requires less power (FIG. 8B). It has a Dewar hull 156 with a volume of less than 100 milliliters. An incoming fluidic line 157 receives sample and carries it through the insulating cap 158, into the fraction collector cartridge 159. Heat is conducted out of the cartridge and enclosure by a flat heat pipe 160. Note that heat pipe is hollow with an internal working fluid, most commonly water. These conductors are move up to 90 times the heat conduction of a solid copper conductor, with significantly less weight. The heat pipe lies adjacent to an external Peltier cooling chip 161 (see magnified view, top right), in contact with the cold side. The hot side is against a vapor chamber 162/heat fin 163 combination. Finally, there is a fan for active heat removal 164, if needed. Temperature control and logging are performed by a temperature sensor 165 and an Internet of Things (IoT) controller 166. No electrical connections are shown, except for the connection to the controller board. A graph (FIG. 8C) shows the cooling performance of the 220 milliliter prototype device at 5.5 Watts power consumption; it reached −13° C. FIG. 8D shows the smaller Dewar jar hull 167, used for the scaled down refrigeration unit shown diagrammatically in FIG. 8B.

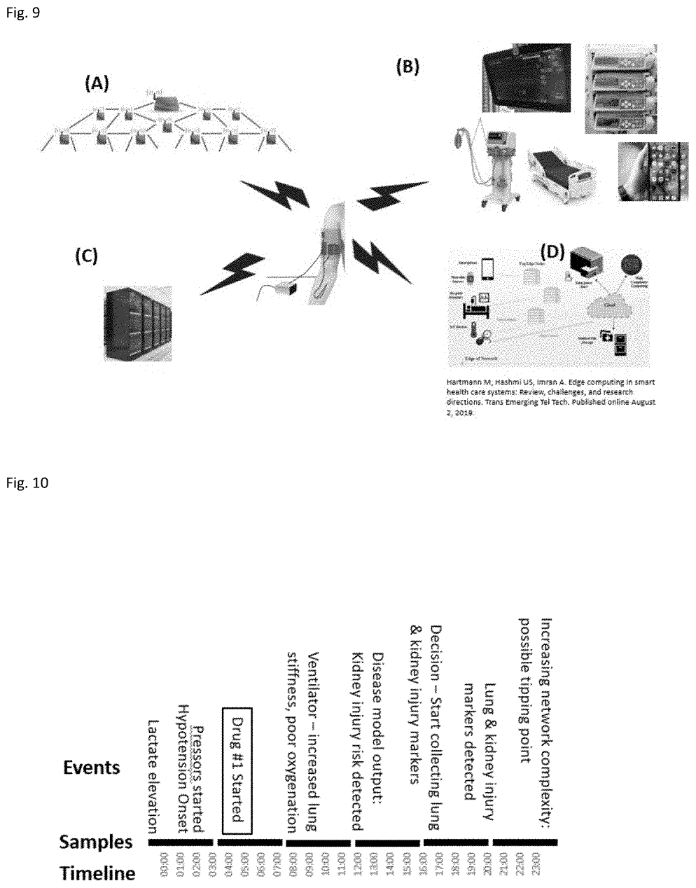

FIG. 9 shows the data gathering functions of the system. Artificial Intelligence and other advanced analytic techniques require large amounts of detailed data that are unnecessary for patient care and would not normally be included in the patient record. Evolving technologies such as IoT sensors and smart devices offer novel approaches to understanding patient condition with nonconventional data. Networks of IoT devices can gather novel data from environment and wearable sensors, using time stamping for event detection (FIG. 9A). Information from smart devices such as mechanical ventilators, intravenous (IV) pumps, bed sensors and inputs from smart phones can augment the molecular data as well (FIG. 9B). The current invention can store and broker this data so that it can be added to the molecular data (both real time and retrospective) for processing by Al and other analytics. Remote server inputs (FIG. 9C) are invaluable for the rich clinical data available in Electronic Medical Records (EMR). They are also valuable for the potential of real time interaction with a beside system like the present invention with data analytics and disease models. This invention can use model output to make sampling decisions, feed back real time molecular and clinical data to the models and test the model's performance at identifying tipping points and molecular events of interest. The edge computing environment (FIG. 9D)—bringing more powerful computing resources into the local network (as opposed to cloud)—allows quicker decisions when needed.

Some examples of data that have shown (or may have) potential in Al but are not systematically collected nor included in the standard EMR include: Data from vibration sensors in hospital beds can detect patient fall risk by patterns of movement. Patient movements can also detect oversedation, undersedation, delirium, escape risk and falls. Weaning patient from mechanical ventilation is a haphazard art, and essential to prevent tracheostomy, chronic long-term care and debilitation. Data from ventilators on lung performance, weaning time, and mode can be used to construct Al optimized weaning. Ventilator data can also detect worsening of lung stiffness seen in acute respiratory distress syndrome (ARDS), as seen in severe COVID-19 and other forms of sepsis. Delirium can be a devastating problem in the critically ill. New monitoring technology such as high density electroencephalography (EEG) and cerebral near infrared spectroscopy (NIRS) may detect early, and categorize delirium. Total body infrared imaging may detect early infection (fever) and peripheral vasoconstriction (hypovolemic shock), unaddressed pain, hypermetabolism (sepsis) etc. There are many other potential non-obvious data sources that can, taken together, expand the data spectrum for data analytics.

In the patient environment, and in any other arrangement where patient data could be at risk, multiple layers of security must be added. There are secure computing strategies wherein predictive disease models work with the patient's individual data as model parameters but never remove the data from the bedside. There are other protocols where patient data is used by a network component, then immediately erased. Many other solutions are being developed.

A concept for a typical data-rich display of the accumulated data that will allow a molecular learning process to begin is shown (FIG. 10).

What is claimed is:

1. An automated computer and fluidic system that characterizes molecular networks in complex systems using data analytics and contextual data to optimize sample collection strategies, to predict important molecular changes, and to confirm them with molecular measurements on obtained samples, comprising:
a networked computer that gathers contextual data and assay results and updates predictive models;
automated fluidic sampling components that store samples for later assays, and also run real time assays;
software controlled valves that split sample stream(s) among storage and assay devices; and
software that creates and stores contextually annotated molecular dynamics databases and constructs an annotated timeline with events of interest and decisions made, for teaching and research purposes.

2. The system of claim 1 further comprising:
Networked computers for obtaining contextual data that may include, but not be limited to, connections with databases, the edge computing environment, cloud computing, Internet of Things (IoT) devices, smart devices, smart phones, and sensors for data input; and
Other network connections to smart devices may include, but not be limited to, patient support devices such as mechanical ventilators, intravenous pumps, cardiac monitors, pulse oximeters, dialysis and extracorporeal membrane oxygenators, hospital bed sensors, patient attached sensors, cardiac assist devices.

3. The system of claim 1 further comprising:
data analytic approaches to be tested that may include, but not be limited to, artificial intelligence algorithms, big data algorithms, dynamic network analysis, clustering techniques, predictive modeling based on both initial and updated conditions, Bayesian and other statistical methods; and
decision software to optimize collection of samples and contextual data that may be based on multi-criteria decision-making or other algorithms.

4. The system of claim 1 further comprising:
software that controls valves to send sample streams to real time assays such as, but not limited to, LOC, POC and other real time analyzers, based on timers or decision-making software;
software that controls valves to send sample streams to sample optimization chambers that contain specific reagents to optimize laboratory or on-chip analysis; and
software that receives the results of real time assays from LOC, POC and other real time analyzers, and incorporates these results into further decisions and an annotated timeline.

5. The system of claim 1 further comprising:
Hardware with active, computer controlled valves, sending samples to storage devices on micro- and milli-fluidic chips;
sample distribution within storage devices managed with passive, hydrophobic valves;
sample protection with superhydrophilic chemistry used extensively on sample contacting surfaces to prevent sample adsorption and absorption;
sample protection within sample storage areas by using superhydrophobic chemistry and limiting this chemistry to only the hydrophobic valves and non-sample contacting areas to prevent sample adsorption and absorption; and
highly absorbent material to contain leaks, preventing personnel or environmental contamination.

6. The system of claim 1 further comprising:
sample storage that includes sample protection and optimization with addition of sample protectants including, but not limited to, protease, DNase, RNase inhibitors, anti-oxidants, anticoagulants, cryoprotectants such as trehalose, sample optimizers, for example ethylenediamine tetraacetic acid (EDTA) for mass spectrometry of lipids and metabolites;
addition of reagents and sample protectants to samples with eductors, separate reagent streams or deposition of lyophilized reagents into wells during manufacture; and
use of surface coatings or bulk device materials that limit the diffusion of gases or water vapor to reduce sample contamination by dissolved gasses or sample volume loss by evaporation.

7. The system of claim 1, further comprising:
sample storage chambers with a tubular geometry, to allow linear filling of sample storage devices to optimize sample filling and emptying avoiding bubbles and sample break up;
strategically placed sensors that detect filling at discrete points in the tubular sample compartments, allowing flow rate calculation and time stamping of sample start and end times for each sample; and
strategic placement of ports to allow automated surface chemistry deposition, and efficient sample removal by automated, high throughput devices.

8. The system of claim 1, further comprising:
computer software that tracks sample movement and other performance metrics to detect system component failure, and issues local and/or remote alarms to system managers; and
computer software that manages a refrigeration module to prevent sample spoilage, along with issuing warnings when temperature exceeds a safe limit.
